(19) United States
(12) Patent Application Publication
(10) Pub. No.: US 2011/0230821 A1
(43) Pub. Date: Sep. 22, 2011

(54) Manual assembly for cardio-circulatory resuscitation

(76) Inventor: Uros Babic, Belgrade (RS)

(21) Appl. No.: 12/932,804

(22) Filed: Mar. 7, 2011

Related U.S. Application Data
(60) Provisional application No. 61/340,764, filed on Mar. 20, 2010.

(51) Int. Cl. A61M 5/31; A61M 1/00; A61M 25/10

(52) U.S. Cl. 604/28; 604/151; 604/35; 604/96.01; 604/101.01

(57) ABSTRACT

In one embodiment a large volume syringe pump assembly with a mechanism for facilitated manual driving (100) is connected to a large bore access cannula (200) which is placed percutaneously into the arrested left ventricular cavity of a victim. The cannula is equipped with balloons for sealing (212) and for immobilization (218). A large bore 3 way stopcock (208) is incorporated into the proximal end of cannula. The syringe pump assembly includes a large volume syringe (140) with a vent for air removal (141). A tubing length (148) is incorporated into the distal end of syringe. Tubing length has a connector at its distal end (150) for rapid connection to cannula. The syringe pump assembly includes a lever (122) pivotal attached to the syringe. By rearward manual movement of the lever the oxygenated blood is aspirated from the left ventricle and from the left atrium into the large volume syringe. The large volume of aspirated blood is vigorously injected back into the arrested left ventricle through the same access cannula (200). Since the large volume of injected blood exceeds the volume capacity of the non-contracting left ventricle, the surplus of injected blood volume is ejected through the aortic valve into the aorta. Perpetuating the aspiration and injection maneuvers by manual actuation of the syringe pump assembly (100) provides perfusion of vital structures during cardio circulatory arrest. Other embodiments are described and shown.

### CROSS REFERENCE TO RELATED APPLICATIONS

This application claims the benefit of provisional patent application Ser. No. 61/340,764, filed 2010 Mar. 20 by the present inventor.

### FIELD OF THE INVENTION

The invention belongs to broad field of urgent and intensive medicine and relates to system for providing of effective perfusion of vital organs during cardio-circulatory arrest in an in-hospital and in out-of-hospital environment.

### BACKGROUND-PRIOR ART

The following is a tabulation of some prior art that presently appears relevant:

### U.S. PATENTS

| Pat. No. | Kind Code | Issue Date | Patentee |
|---|---|---|---|
| 7,131,953 | B2 | 2006-07-11 | Scherman |
| 5,399,148 | B2 | 1995-03-21 | Waide |
| 6,406,422 | B2 | 2003-01-28 | Landesberg |
| 5,190,528 | B2 | 1993-03-02 | Fonger |
| 7,494,477 | B2 | 2009-02-24 | Rakhorst |
| 7,524,277 | B2 | 2009-04-28 | Wang |
| 3,952,742 | B2 | 1976-04-27 | Taylor |

### FOREIGN PATENT DOCUMENTS

| | Cntry | | | |
|---|---|---|---|---|
| Foreign Doc. Nr. | Code | Kind Code | Pub. Dt. | App or Patentee |
| WO94/03228 | EP | A3 | 1994-02-17 | Zadini |
| WO 98/05289 | WO | A1 | 1998-02-12 | Fogarty |

### NON PATENT LITERATURE DOCUMENTS

De Souza C F. et al. Percutaneous Mechanical Assistance for The Failing Heart. J Interven Cardiol 2010; 23:195-202.
Arlt M, Phillipp A, Zimmermann M, Voelker S, Hilken M, Hobbhahn J, Schmid Ch. First experience with a new miniaturized life support system for mobile percutaneous cardiopulmonary bypass. Resuscitation 2008; 77: 345-350.
Chen S I, Ko W J, Lin F Y. Insertion of Percutaneous ECMO Cannula. Am J Emerg Med 2000; 18:184-185.
Webb J, The shortest Way to the Heart. Catheterization and Cardiovascular Interventions 2008; 71:920.
Turgut T, Deeb M, Moscucci M, Left ventricular apical puncture: A procedure surviving well into the new millennium. Catheterization and Cardiovascular Interventions 2000; 49:68-73.
Walters D L. et al. Catheter Cardiovascular Intery 2003; 58:539-44: Trans thoracic left ventricular puncture for assessment of patients with aortic and mitral valve prosthesis: Massachusetts General hospital experience 1989-2000.
Mihaylov D. et al. Evaluation of the optimal driving mode during left ventricular assist with pulsatile catheter pump in calves. Artificial organs 1999; 23:1117-1122.
International Liaison Committee on Resuscitation. Part 2.Adult basic life support. Resuscitation 2005; 67 (2/3):187-201.

Acute cardio-circulatory arrest is the most frequent cause of sudden death. Emergency treatment includes artificial ventilation and external cardiac massage with chest compressions. Treatment of cardio-circulatory arrest has not changed since 50 years. Electrical cardiac instability is effectively treated with defibrillations while pulmonary function is replaced by mechanical ventilation after placement of tracheal tube. Mechanical chest compression is aimed at maintaining perfusion of vital organs. Low success rate of resuscitation measures is mainly attributed to ineffectiveness of chest compressions to provide minimum of needed perfusion. The pressure differences between aorta and right atrium as the principal determinant of existing circulation, is negligible during chest compressions. Numerous versions of mechanical apparatus for external cardiac compression have emerged: U.S. Pat. No. 7,131,953 to Scherman (2006) shows a resuscitation device for automatic compression of a victim's chest using a compression belt operable attached to a platform on which a victim rests. U.S. Pat. No. 5,399,148 to Waide (1995) shows an external cardiac massage device comprising a pressure source and depressor means for adjustable cardiac compression. Numerous versions of apparatus for internal cardiac massage also emerged: W.O. Pat. No. 94/03228 to Zadini (1994) shows an apparatus comprising an expandable member placed inside the chest adjacent the heart whereby during inflation of the expandable member the heart is compressed between the thoracic spine and the member itself; W.O Pat. No. 98/05289 to Fogarty (1998) shows a minimal invasive direct cardiac massage device comprising an inflatable bladder introduced through the intercostals space and placed between the sternum and the heart.

None of these numerous versions of mechanical apparatus for external or internal cardiac compression has substantially improved the effective blood flow during cardio-circulatory arrest.

In specialized hospitals, sporadically, systems for artificial mechanical circulation are used: accessing a reservoir of blood (cardiac chamber), withdrawal of the blood into a pump and returning of the blood into the circulation with pump energy. U.S. Pat. No. 5,190,528 to Fonger (1993) shows system of cannulas for accessing the left atrium by so called transseptal route: Under fluoroscopy a cannula is placed from the groin vein transseptally into the left atrium and the oxygenated blood from the left atrium is transported with a roller pump through the second cannula placed through the groin artery into aorta. Installation of this system requires cardiac catheterization team and equipment. U.S. Pat. No. 7,494,477 to Rakhorst (2009) shows a pulsatile catheter that is introduced through the aorta retrograde across the aortic valve and placed into the left ventricle. The blood is drained from the left ventricle and pumped back into the aorta through the same catheter. The pulsatile 25Fr catheter with an unidirectional valve situated inside the catheter lumen between distal opening within the left ventricle and side-opening situated in the aorta, and the extracorporeal displacement chamber with a volume of 60 ml provides a flow of up to 2.9 lit/min. (Mihaylov D. et al. Evaluation of the optimal driving mode during left ventricular assist with pulsatile catheter pump in calves. Artificial organs 1999; 23:1117-1122.). This system is aimed at assisting a failing heart and cannot replace the pumping function in case of arrest. Also its implementation is complex requiring surgically created access to aorta or a great artery. DE Souza C F. et al describe in their paper (DE Souza C F. et al. Percutaneous mechanical Assistance for The Failing Heart. J Interven Cardiol 2010; 23:195-202) today's most usable systems for nonsurgical percutaneous mechanical circulation. Intraaortic balloon pump is useful only as a support to failing heart, it is not useful during cardiac arrest. Hemopump™=turbine pump placed on the tip of the access cannula introduced through a groin artery retrograde into the left ventricle wherefrom it transports the blood to aorta. This system is aimed at supporting a failing heart although sporadically it has been used during cardiac arrest too. For its placement a fluoroscopy is needed, vascular access is a problem, pumping blood volume capacity is low and it produces aortic pressure of up to 50 mmHg. The percutaneous left ventricular assist device “Tandem Heart” is a left atrial to femoral artery bypass having a 21 Fr left atrial cannula for withdrawing of the oxygenated blood from left atrium which is then injected by means of a centrifugal pump into the femoral artery establishing the bypass.

This system provides a flow of up to 4 L/min. Also this system is aimed at assisting a failing ventricle and not for replacing an arrested pumping function.

Cardio-pulmonary support system comprising access cannula for accessing large central systemic veins wherefrom the blood is withdrawn into a console with oxygenators (apparatus which replaces the lung) after which the blood is pumped back into the aorta through the cannula placed through a groin artery. A miniaturized portable version of cardio-pulmonary support is available (Arlt M, et al. First experience with a new miniaturized life support system for mobile percutaneous cardiopulmonary bypass. Resuscitation 2008; 77: 345-350). This system is expensive, complex, and requires a specialized trained team for its utilization.

Also for its implementation in average 30 minutes are needed (Chen S I, Ko W J, Lin F Y. Insertion of Percutaneous ECMO Cannula. Am J Emerg Med; 2000; 18:184-185).

Advantages of cardio-pulmonary systems are large blood volume pumping capacity of up to 6 Lit/min, disadvantages of this system are—the lung and the left heart remain without circulation—they are not decompressed; often blood transfusions are needed, for installation of this system a well trained team is needed and time needed for its implementation, which time is not available in urgent situations during cardiac arrest. All described systems for percutaneous mechanical circulation require access through the large veins and/or arteries.

A heart chamber is filled with blood and it has larger volume than any groin artery or vein and thus it can be easier punctured especially during cardiac arrest. Percutaneous transthoracic puncture of the left ventricle has been utilized for diagnostic purposes in patients with implanted metallic heart valves in aortic and mitral position. Complication rates were acceptable (Walters D L. et al. Catheter Cardiovascular Intery 2003; 58:539-44: Transthoracic left ventricular puncture for assessment of patients with aortic and mitral valve prosthesis: Massachusetts General hospital experience 1989-2000). Percutaneous transthoracic access has been used recently for catheter based procedures like closure of paravalvular leaks (Webb J, The shortest Way to the Heart. Catheterization and Cardiovascular Interventions 2008; 71:920; Turgut T, Deeb M, Moscucci M, Left ventricular apical puncture: A procedure surviving well into the new millennium. Catheterization and Cardiovascular Interventions 2000; 49:68-73). U.S. Pat. No. 7,524,277 to Wang (2009) shows a system that utilizes a single transapical entry site into the left ventricle for placement of a single cannula with a common access to left ventricle and to aorta. This system obviates the need for two access sites since blood is withdrawn from the left ventricle through the larger outer part of cannula and injected back into the aorta through the smaller inner cannula with the tip of smaller cannula situated in the aorta. Blood aspiration through the outer part of cannula and blood injection through the central part of cannula by external driving apparatus needs a large diameter of the single entry site and a safety regulation adjusted to available blood volume. Also, this system for assisting a heart is complex for an emergency need during a cardiac arrest.

Access to heart chambers by thoracoscopic methods is described in US Pat. No. 3,952,742 to Taylor (1976): a transthoracic cannula is equipped with penetration needle and with two axially spaced balloons for stabilization and sealing purposes; this resuscitation transthoracic cannula is aimed at providing electrical support to an arrested heart as well as at providing an application of needed medication during resuscitation. This system however provides no circulatory support.

U.S. Pat. No. 6,406,422 B1 to Landsberg (2003) shows a system for assistance of failing heart (assist device) that utilizes a single cannula for drainage out of heart chamber and for return of blood into the chamber of a failing heart (single cannula ventricular assist apparatus). This system is aimed at supporting a failing heart chamber (usually left ventricle) in such a way that it uses the computer regulated withdrawal of the small amount of blood out of the left ventricle during the diastole and also computer regulated return of the small amount of the blood back into the left ventricle during ventricular systole. This method is used to augment the existing stroke volume of a failing heart.

The blood volume added with this system during ventricular native contraction is small—about 30 ml per systole. This system is not suitable for replacement of heart pump function during a cardiac arrest.

Based on the prior art analysis there is obviously need for a system, method, device that could enable an effective perfusion of vital structures (brain and heart muscle) during cardiac arrest. Such a device should have following characteristics: it should be implementable within a short period of time (e.g. within 3 minutes); it should be applicable at any place were a victim could be located; it should be of small size in order to fit into a first aid case and thus affordable to any emergency first aid medicine professionals in hospital as well as in out of hospital environment.

### SUMMARY

The aim of this invention is to provide a system of rapid establishment of circulatory flow during a cardio-circulatory arrest in an in-hospital and in an out-of-hospital environment, a system simple to use and less expensive in order to be affordable to majority of professionals involved in resuscitation activities.

In accordance with one embodiment, a device system for invasive resuscitation of arrested circulation includes a large bore cannula for accessing an arrested cardiac chamber and a large volume syringe attached to a frame. The syringe includes a mechanism for power facilitated manual driving. The access cannula includes means for introduction by over the wire introducing technique. In one embodiment the cannula is equipped with two balloons for sealing and stable anchorage within the intracorporeal passageway. The large volume syringe and access cannula are interconnected by a large bore 3-way stopcock. In one embodiment, after transthoracic placement of the access cannula into the arrested left ventricle, the cannula is connected to the syringe and the oxygenated blood is manually aspirated from the left ventricle and from the left atrium and rapidly injected back through the same cannula into the left ventricle. The system utilizes the naturally existing two unidirectional check valves within the arrested left heart: mitral inflow check valve, and aortic outflow check valve.

Application of negative aspiration pressure opens the inflow check valve and closes the outflow check valve allowing drainage of the whole left heart.

Aspiration volume=left ventricular volume+left atrial volume.

During a rapid injection of large blood and/or fluid volume into the arrested left ventricle the pressure inside the left ventricle rises and closes the inflow check mitral valve. Since the injecting volume exceeds the volume capacity of the arrested left ventricle the blood surplus is injected across the outflow check valve into aorta.

Injecting stroke volume=total injecting volume−volume capacity of the arrested left ventricle.

Assuming a left atrial volume of 80 ml, a left ventricular volume of 100 ml the maximal aspirating volume would be 180 ml. Assuming an injecting volume of 180 ml by known left ventricular volume capacity of 100 ml the injecting stroke volume would be 80 ml.

### BRIEF DESCRIPTION OF THE DRAWINGS

FIG. 1A shows the syringe pump assembly with double handed driving connected to transthoracic cannula.

FIG. 1B shows the large syringe of the double handed device.

FIG. 1C shows the frame into which large syringe is placed during use.

FIG. 2A is a perspective view of the transthoracic access cannula assembly with balloons deflated and with introducer dilator with guiding wire ready for introduction.

FIG. 2B is a perspective view of the transthoracic access cannula with inflated balloons.

FIG. 2C is a perspective close view of distal end of transthoracic cannula with inflated balloons.

FIG. 2D is a perspective view of the access cannula placed across an introduction splittable introducer sheath.

FIG. 3A is a perspective view of the device in place during aspiration.

FIG. 3B is a perspective view of the device in place during injection.

FIG. 4A shows a transvascular cannula assembly with introducing dilator and pigtail catheter with guiding wire.

FIG. 4B shows transvascular cannula without introducers.

FIG. 4C shows transvascular cannula with introducers in place.

FIG. 5A shows double handed manual device with transtvascular cannula

FIG. 5B shows the system with transvascular cannula in place introduced through axillary/subclavian artery.

FIG. 5C. shows the system with transvascular cannula in place introduced through a groin artery.

FIG. 6A shows the syringe pump assembly with lever driving mechanism in aspiration position.

FIG. 6B shows the pump assembly with lever driving in injection position.

FIG. 6C shows the frame used for device with lever driving

FIG. 6D shows the syringe with fasteners used for device with lever driving.

FIG. 6E shows syringe pump assembly with lever driving.

FIG. 7A shows the device with lever driving pump assembly connected to transthoracic cannula

FIG. 7B shows the device with lever driving and with an added infusor utilized by an aid for resuscitation of a victim.

FIG. 8A shows double syringe pump assembly with lever driving and with transthoracic cannulas.

FIG. 8B shows double syringe pump assembly with lever driving and with access cannulas in place.

FIG. 9A shows double syringe pump assembly with lever driving connected to double transvascular cannulas.

FIG. 9B shows double syringe pump assembly with lever driving connected double transvascular cannulas introduced into right and left ventricle through subclavian vein and axillary-subclavian artery respectively.

FIG. 9C shows double syringe pump assembly with lever driving connected double transvascular cannulas introduced into right and left ventricle through the left and right groin vein respectively.

FIG. 9D shows double syringe pump assembly with lever driving connected double transvascular cannulas introduced into right and left ventricle through groin vein and groin artery respectively.

FIG. 10A shows double syringe pump assembly with lever driving connected transvascular cannula and transthoracic cannula.

FIG. 10B shows double syringe pump assembly with lever driving connected transvascular cannula and transthoracic cannula introduced into right and left ventricle through subclavian vein and through left ventricular apex respectively.

FIG. 11 shows an additional embodiment with manual and alternatively with motorized actuation.

### REFERENCE NUMERALS

| | | | |
|---|---|---|---|
| 100′ | double handed syringe pump | 109′ | mechanical stop on proximal |
| | assembly | | end of syringe |
| 102′ | top entrance of frame | 124′ | double handed grip |
| 103′ | frame to syringe fasteners | 126′ | mechanical props |
| 106′ | base of frame | 127′ | holes for fasteners on mechanical |
| | | | stop |
| 128′ | side hole on cylinder frame | 106 | base of frame |
| | for exit of distal syringe end | 107 | removable arcuate syringe |
| 129′ | cylinder frame | | fastener to frame |
| 131′ | folding pedal | 108 | beveled part of frame |
| 133′ | pivotal connection pedal to | 110 | flange |
| | base of frame | 112 | pivot pin lever to flange |
| 140′ | syringe for double handed pump | 120 | spaced leg of lever |
| 142′ | piston | 121 | second spaced leg of lever |
| 144′ | plunger | 122 | lever |
| 148′ | tubing length of syringe | 122b | bolt on lever |
| 150′ | connector of syringe to cannula | 123 | bridge |
| 100 | syringe pump assembly | 123n | nut inside bridge for bolt of lever |
| | with lever driving | 124 | grip |
| 102 | top plate of frame | 100″ | doubled syringe pump assembly |
| 103 | fixed arcuate syringe fastener | 125 | removable connector |
| | to frame | | for common grip |
| 104 | vertical arm of frame | 124″ | common grip for two pumps |
| 105 | rod connector of fasteners | 130 | leg of plunger's holder |
| 105b | bolt on end of rod connector | 132 | pivot pin -lever to plunger's holder |
| 105h | hole on fixed fastener | 134 | fastener of plunger's holder to legs |
| | for rod connector | 136 | fastener for plunger's holder |
| 105s | screw for fastening of rod | | to legs. |
| | connector to fixed fastener | 138 | plunger holder |
| | | 138n | nut inside plunger holder |
| 140 | large volume syringe for | 210 | distal end opening of the cannula |
| | lever driving assembly | 211 | side hole |
| 141 | vent tube for air removal | 212 | larger (outer) compliant balloon |
| 142 | piston | 214 | port for inflation and deflation |
| 143 | small 3 way stopcock for | | of larger balloon |
| | air vent tube | 215 | syringe for inflation and deflation |
| 144 | plunger | | of larger balloon |
| 144b | bolt on proximal plunger for | 216 | channel for inflation and deflation |
| | fastening to plunger holder | | of larger balloon |
| 145 | opening for vent tube | 218 | smaller (inner) noncompliant |
| 146 | mechanical stopper for piston | | balloon |
| 147 | muff incorporated on syringe | 219 | proximal flat part of smaller |
| 148 | tubing length of syringe | | noncompliant balloon |
| 150 | tubing length connector -to cannula | 220 | port for inflation and deflation |
| 200 | transthoracic cannula | | of smaller balloon |
| 200″ | double transthoracic cannula | 222 | channel for inflation and deflation |
| | arrangement | | of smaller balloon |
| 202 | braided part of cannula shaft | 224 | syringe for inflation and deflation |
| 204 | braided part of cannula shaft | | of smaller balloon |
| | with thicker shaft wall | 230 | introducing large dilator |
| 206 | tubing length integrated | 232 | distal tapered end of the large |
| | into cannula shaft | | dilator |
| 208 | large bore 3-way stopcock | 233 | distal large dilator's port for |
| | integrated into proximal end | | guiding wire |
| | of cannula | 234 | proximal end of the large dilator |
| 235 | proximal large dilator's port | 310 | introducing dilator of transvascular |
| | for guiding wire | | cannula |
| 240 | guiding wire | 312 | distal tapered tip of dilator |
| 242 | distal J shaped end of guiding wire | | for transvascular cannula |
| 250 | splittable introducing sheath | 314 | proximal end of dilator for |
| 252 | distal end of splittable sheath | | transvascular cannula |
| 254 | proximal arm of splittable sheath | 316 | proximal dilator port for pigtail |
| 255 | second proximal arm of | | catheter |
| | splittable sheath | 318 | distal dilator port for pigtail catheter |
| 300 | transvascular cannula | 320 | pigtail catheter |
| 302 | braided part of transvascula cannula | 322 | distal end of pigtail catheter |
| 304 | proximal non braided wider part | 324 | proximal end of pigtail catheter |
| | of transvascular cannula | | with port for guiding wire |
| 328 | large bore 3 way stopcock integrated | 326 | long guiding wire |
| | into proximal end of transvascular | 400 | infusor |
| | cannula | 500 | removable connector of motor arm |
| 306 | distal end opening of transvascular | | to plunger of the syringe |
| | cannula | | |
| 308 | side holes of transvascular cannula | | |

### ABBREVIATIONS AND GLOSSARY

AV aortic valve
IVS interventricular septum
LA left atrium
LV left ventricle
MV mitral valve
RA right atrium
RV right ventricle
TV tricuspid valve
TRANS THORACIC refers to an access directly through the chest wall and chest cavity
TRANSVASCULAR refers to an access through the wall of a blood vessel
PROXIMAL refers to a location close to the operator or actuator
DISTAL refers to a location remote from the operator or actuator
INTRACORPOREAL refers to location within a body
STERNOTOMY refers to cutting through the chest bone after which the chest bone itself is divided
INTERVENTRICULAR SEPTUM refers to wall between right and left heart chamber
ECMO refers to extracorporeal membrane oxygenator-apparatus for oxygenation of blood outside body
SELDINGER technique refers to introducing a catheter-like items over the wire into a blood vessels or into a hollow organ without surgically created entry site

### DETAILED DESCRIPTION

### First Embodiment

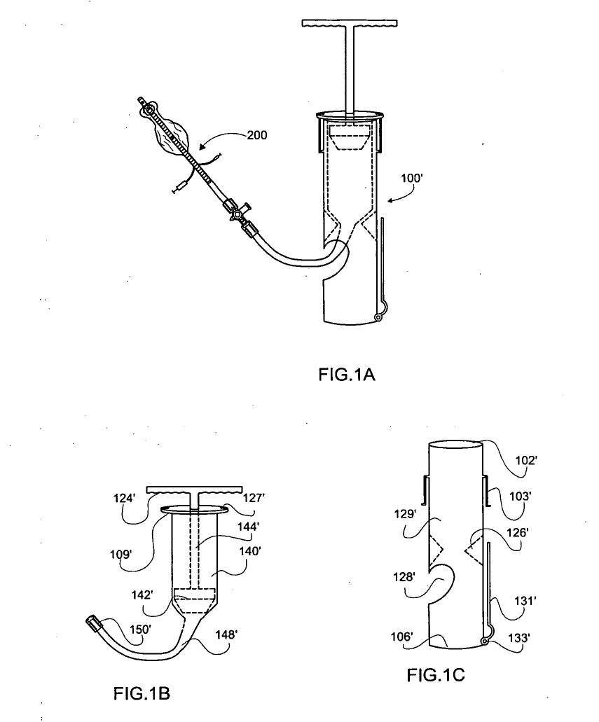

### FIGS. 1-3

FIG. 1A shows the manual device with double handed driving for cardio circulatory resuscitation in accordance with described embodiment that has syringe pump assembly 100′ and transthoracic cannula 200. The device may have a total weight of less than 3 kg.

FIG. 1B shows the large volume syringe 140′ that has a piston 142′ and a plunger 144′ with a grip 124′ for double handed power actuation. Syringe 140′ continues distal as a tubing length 148′ that ends with a connector 150′. Syringe 140′ is made of medical polymer while its distal part tubing length 148′ is made of polyvinyl chloride. There is mechanical stopper 109′ on proximal end of syringe. Stopper 109′ has two holes 127′ for accepting the fasteners 103′ of the frame cylinder 129′. Frame cylinder 129′ has top entrance 102′ for syringe, side hole 128′ for exit of distal syringe part. There is a pedal 131′ attached pivotally 133′ to the base of frame 106′. Frame 129 may be made of transparent firm plastic. Pump assembly 100′ can be provided as a sterile compact unit ready for use so that no time is needed to set up.

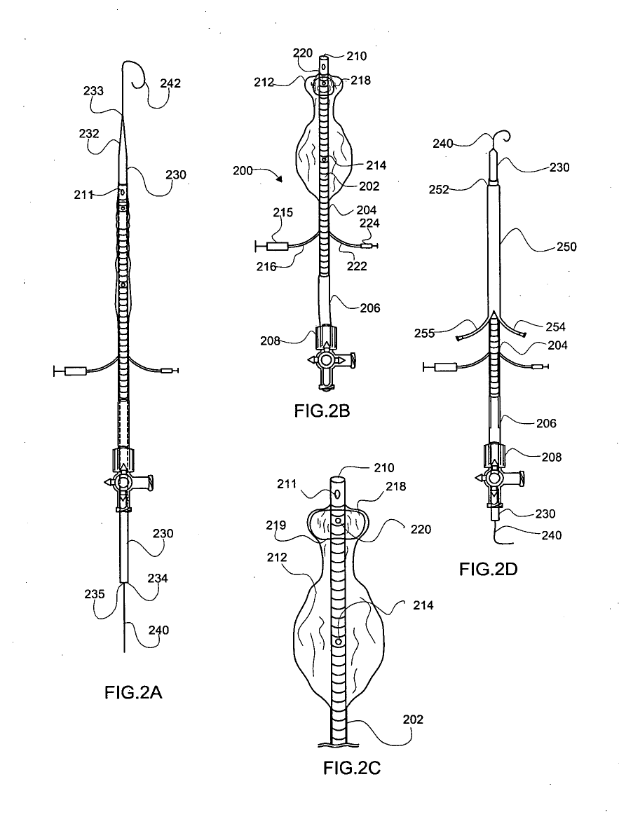

FIGS. 2A-C show the transthoracic cannula assembly. The shaft of the large bore cannula 200 has a braided part 202, a braided part with a thicker wall 204, and a proximal non braided part which is a tubing length integrated into cannula shaft 206. Cannula 200 may be of different sizes for pediatrics and adults e.g. 2-8 mm inner lumen, (6-24 Fr). Part 204 has a thickened shaft wall for accommodating the channels for inflation/deflation of balloons. A 3-way large bore stopcock 208 is incorporated into the proximal end of the tubing length 206. Stopcock 208 has the same large tubular lumen as the cannula 200 in order not to reduce the flow capacity of the cannula and in order to allow the passage of the introducing large dilator 230 during introduction. Cannula 200 has a distal end opening 210 and side holes 211 allowing high flow. The inner surface of cannula 200 and the stopcock 208 have an antithrombotic Heparin coating. Close to the distal end of cannula a non compliant smaller balloon 218 is incorporated into the external part of the shaft wall. There is a port 220 for inflation and deflation of the balloon through the channel 222 by a syringe 224. Balloon 218 can be made of medical polymer like non-compliant polyurethane. The balloon size and shape is appropriate so as to preventing the cannula to be expelled out of the cardiac chamber during vigorous injection. Its proximal aspect 219 is expanded in a direction vertical to the shaft long axis which should prevent a sliding of the balloon back across the entry site. There is a second larger balloon 212 incorporated distally, over the smaller balloon 218, to the distal portion of the shaft 202 and attached proximally onto the proximal portion of the shaft 204. Balloon 212 is made of compliant polymer like compliant membrane of polyurethane, it is inflated and deflated trough the channel 216 by a syringe 215 and through the port 214. Balloon 212 exerts a low pressure making a waist within the passageway through the heart chamber wall and through the chest wall, it expands distally within the cavity of heart chamber and proximally out of body in front of the entry site through the chest wall. Balloon 212 covers the whole intracorporeal passageway of the access cannula providing a sealing. Balloons 218 and 212 together provide a stable anchorage of the cannula. The cannula is introduced directly through the chest wall together with a large dilator 230 over a guiding wire 240. The inner lumen of dilator 230 accepts the guiding wire, there is a proximal port for guiding wire entrance 235 and a distal port 233 for exit of wire 240, dilator 230 has a tapered distal end 232 for easier passage through the chest- and heart wall.

FIG. 2D shows cannula introduction through a splittable introducing sheath 250 having distal end 252 and proximal two arms 254 and 255 for splitting and removal after inserting the cannula. The splittable sheath is introduced together with cannula 200 and dilator 230 over the wire 240. Cannula 200 can then be introduced through the splittable sheath 250 after which the sheath 250 can be removed by splitting it lengthwise. This alternative way of introduction may protect the balloons during introduction however it might be more time consuming.

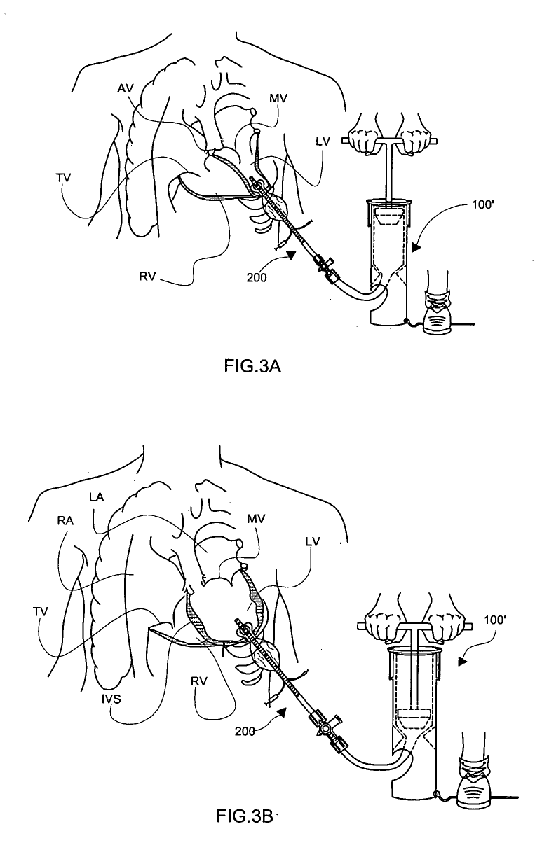

FIG. 3A shows the device assembly with double handed driving in place during an aspiration phase. An aid actuates the pump assembly with both hands and immobilizing it by foot. The inflow check valve-mitral valve is open allowing the blood drainage from the whole left heart and possible even from the pulmonary veins. The aortic valve is closed due to negative aspiration pressure. The large compliant balloon seals the whole intracorporeal passage way of the access cannula, while the small noncompliant balloon inside the left ventricle provides safety against rearward dislodgement. The left ventricular cavity is reduced. The right ventricular cavity is enlarged due to leftwards movement of interventricular septum, the tricuspid valve between the right atrium and the right ventricle is open.

FIG. 3B shows the device assembly in place during an injection phase. The mitral inflow check valve is closed due to increased left ventricular pressure, the outflow check valve i.e. aortic valve is open due to increased pressure in front of the valve and the surplus of the injected blood volume is expelled into the aorta. The left ventricular cavity is significantly expanded while the right ventricular cavity is reduced due to rightwards movement of interventricular septum. FIGS. 3 A and B illustrate the effects of the device utilization: the mechanical pump function of the left heart is regulated by hydraulically transmitted energy created by power manual driving of the syringe pump 100′. The heart wall expands and contracts while the heart valves open and close in accordance with pressure and volume changes within the arrested chamber. The right ventricular cavity changes also in accordance with significant movement of interventricular septum.

### Operation

### FIGS. 3A-B

The device in accordance with described embodiment can be used as an alternative to prolonged chest compression or as a last resort measure after failure of traditional resuscitation attempts.

If a professional aid has exhausted all available measures to restore spontaneous circulation in a victim, this device assembly should be used:

puncturing of the left ventricular apex with a vascular needle through the fifth intercostals space at midclavicular line. The puncture does not need any imaging guidance; aspirating a small amount of red colored blood indicates that the left ventricle is punctured (during a resuscitation the oxygen saturation within the left heart is >90% and within the right heart is <30% which gives a visible color differences in blood samples—a blood sample from left heart is red colored while a blood sample from the right heart is dark colored “Ward K R. Barbee W, Ivatury R R. Monitoring techniques during CPR in “Cardiopulmonary Resuscitation 2000: Chapter 28:480-482. Editors Ornato J P, Peberdy M A, Humana Press Inc. Totowa, N.J. 07512”);
injecting of medication against clotting (Heparin) through the puncturing needle into the punctured heart chamber;
placing of a J guiding wire 240 through the needle; after removal of the puncture needle the cannula assembly (FIG. 2A) with introducer dilator 230 is introduced per Seldinger technique over the wire 240 into the left ventricle;
after advancement of the cannula into the heart chamber the smaller non compliant balloon 218 is inflated by fluid injection through the channel 222 and the cannula is retracted back over the dilator 230 until a resistance is felt which indicates that balloon 218 is contacting the inner wall of the heart chamber (this will indicate the appropriate good placement of the cannula); dilator 230 is removed together with guide wire 240; the stopcock 208 is closed; then the larger outer compliant balloon 214 is inflated by injecting fluid through the channel 216 this will provide sealing and stabilization of the cannula within the intracoerporeal passageway; the syringe pump assembly 100′ is connected to the access cannula 200 by the stopcock 208; stopcock 208 is opened and the pump assembly is actuated double handed by pulling the plunger 144′ upwards which provides aspiration of the oxygenated blood from the left ventricle and from the left atrium and even from pulmonary veins; next stopcock 208 is closed and directed to enable removal of air bubbles if any existing; stopcock 208 is directed to free the flow from the syringe 140′ into the access cannula 200; the aspirated blood volume is vigorously injected back through the cannula 200 into the left ventricle (FIG. 3B); aspiration and injection maneuvers are repeated as long as necessary. Manual driving enables application of aspiration pressure adjusted to the available blood volume within the left heart. Manual driving enables adequately high pressure during injection. There is no need to keep the rate of aspiration and injection too high. It is important to provide sufficient blood and fluid volume to systemic circulation and to keep the mean aortic pressure at an acceptable level. The aspiration phase should be longer and the injection phase shorter.

The system is immobilized by holding the foot on the pedal during operation.

The manual driving enables blood withdrawal adjusted to the available blood volume within the left heart avoiding tubing collapse and or aspiration of surrounding tissue and/or aspiration of extracorporeal air alongside the passageway of the access cannula. The right heart functions like a passive conduit having two unidirectional valves which will be open when the central venous pressure is higher than the pressure within lung vessels, as in Fontan's circulation

(The “Fontan's circulation” refers to the configuration where the single ventricle pumps blood returning from the lungs to the body, and the blood returning from the body travels to the lungs by direct blood vessel connections without a pumping chamber).

It is important to increase the volume within the central venous system which can be done e.g. by passive rising of victim's legs.

Should there be not enough aspirating blood volume, additional fluid volume could be aspirated from the fluid infusion attached to the sidearm of stopcock 208.

Also additional medication against clotting (Heparin) is added through the side-arm of stop-cock 208.

Alternatively, the access cannula can be introduced with a splittable introducing sheath 250 (FIG. 2D). The splittable sheath together with cannula 200 and dilator 230 is introduced over the wire 240. Sheath 250 is removed by splitting it lengthwise leaving the cannula in place and the procedure proceeded as described.

After restoration of spontaneous circulation pump assembly 100′ can be disconnected leaving the access cannula 200 in place with infusion attached to the cannula. After termination of the support the cardiac access site could be closed with a myocardial free wall occluder or surgically.

### Alternative Embodiment

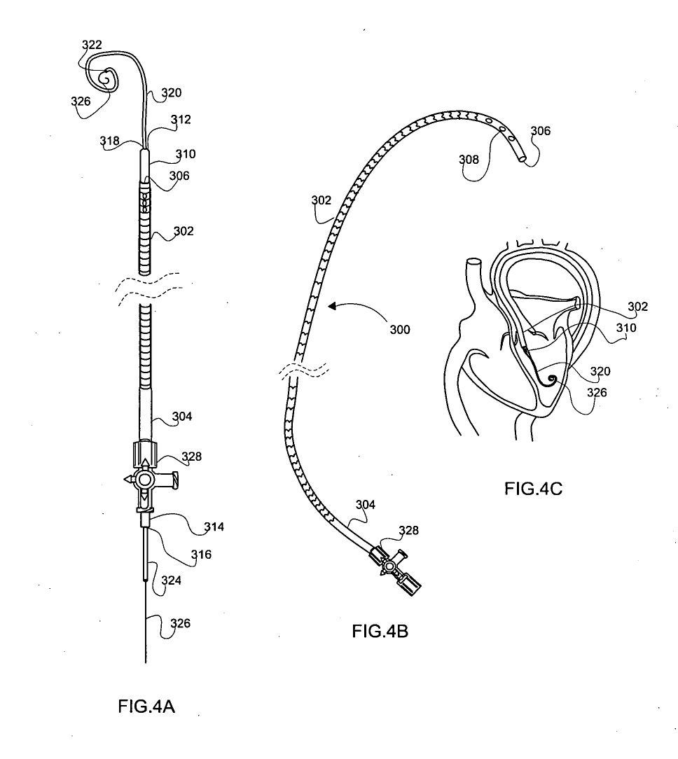

### FIGS. 4A-C, 5A

FIGS. 4A-C show a transvascular cannula assembly for introduction through the wall of a blood vessel. Transvascular cannula 300, with a lumen large enough to enable adequately high flow, has a longer braided part 302, a shorter proximal non braided part 304, distal end opening 306, side holes 308, large bore 3-way connector 328 is integrated into proximal end of proximal part of transvascular cannula 304. Cannula assembly includes an introducing dilator 310 with tapered tip 312 with a proximal end 314 that has a proximal port for pigtail catheter 316, and a distal port for pigtail catheter 318 through which a pigtail catheter 320 can pass. The pigtail catheter 320 has a distal tapered end 322 and a proximal end with port 324 for a long guiding wire 326.

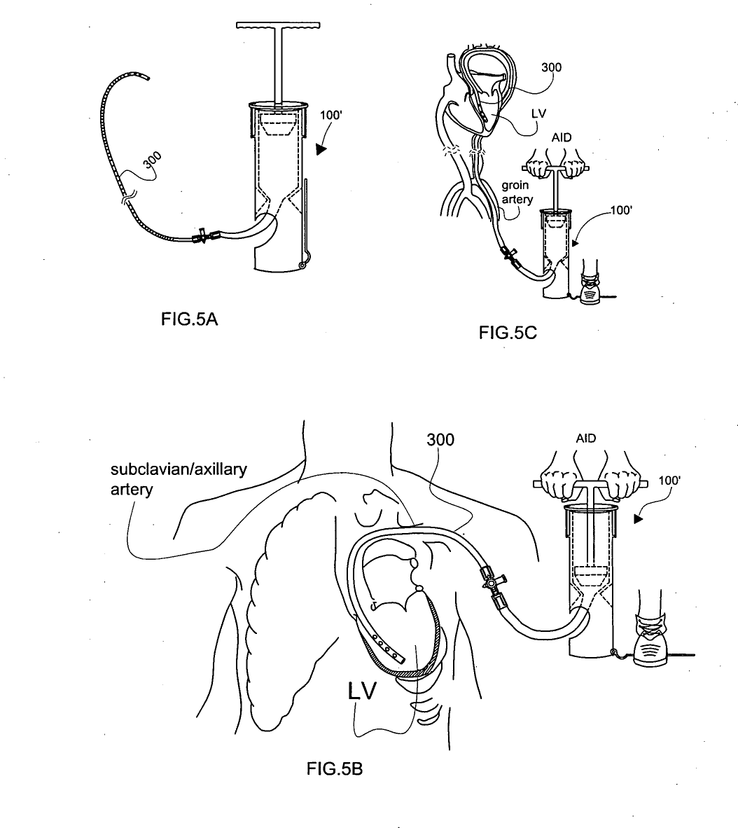

FIG. 5A shows the device in accordance with described alternative embodiment that has a syringe pump assembly 100′ connected to transvascular cannula 300.

### Operation

### FIG. 5B

Puncture of subclavian/axillary artery with a vascular needle; advancement of the long guiding wire 326 through the needle, removal of the needle; advancement of the transthoracic cannula assembly with pigtail catheter 320, and dilator 310 over the wire, retrograde through the aortic valve until the cannula is situated within the left ventricle; removal of wire 326 and removal of introducing dilator 310 with pigtail catheter 320, and connection of the cannula 300 to pump syringe assembly 100′; manual actuation of the system as described before.

This alternative embodiment can be used mostly in an in-hospital environment where there are some tools for guiding the procedural activities like ultrasound or fluoroscopy. FIG. 5C shows described alternative embodiment in place utilizing an installation through a groin artery.

### Alternative Embodiment

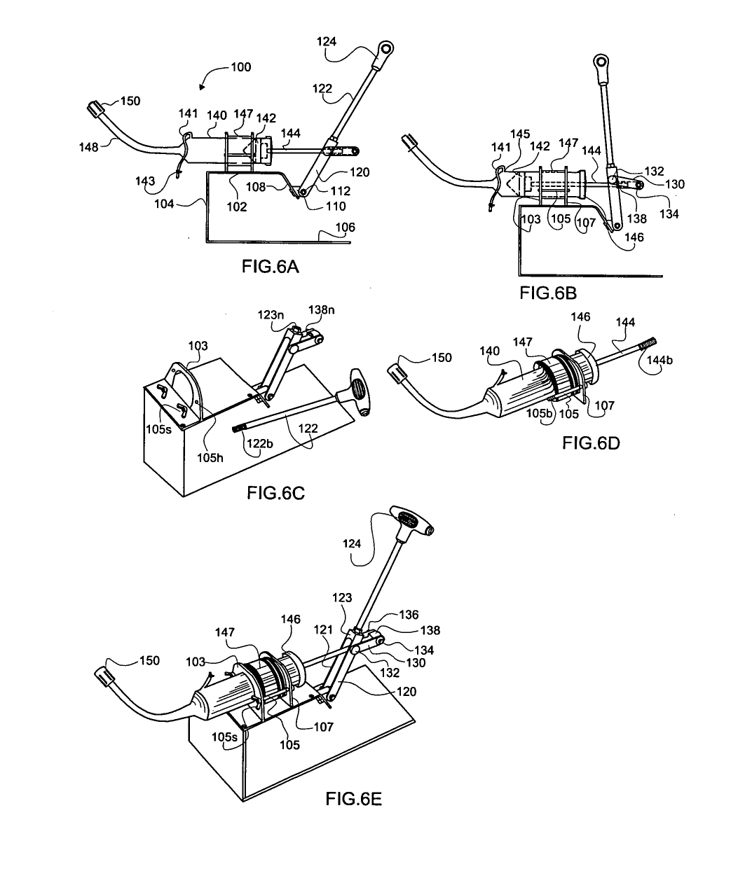

FIGS. 6 (A-E) show a syringe pump assembly with lever mechanism 100 constructed in accordance with one embodiment. A large volume syringe 140 having a plunger 144 with piston 142 is attached onto the top plate 102 of frame. Syringe 140 is made of firm medical polymer. The inside surface of the syringe can be covered with a substance against clotting (heparin coating). Syringe 140 has a large volume capacity (e.g. more than 200 ml) that significantly exceeds the volume capacity of an arrested heart chamber. The frame consists of top plate 102, base 106, a vertical part 104 and beveled part 108. There is a flange 110 on the frame (FIG. 6A). A lever 122 includes two spaced legs 120 and 121 interconnected by bridge 123 (FIG. 6E). Legs 120 and 121 are attached by pivot pin 112 to the flange 110 (FIG. 6A). Pivot attachment enables forward and rearward movement of the lever. Lever 122 has a grip 124 at the top. The longer part of lever 122 with grip 124 may be removable from the legs 120 and 121 by bolt on lever 122b which is connected to nut 123n inside bridge 123 (FIG. 6C). The tubing length 148 is integral part of distal end of syringe. Tubing length 148 is made of polyvinyl-chloride and has the inner surface coated with heparin. There is tubing—to cannula connector 150 on the distal end of the tubing. There are two additional spaced legs 130 attached distally to the lever spaced legs 121-120 by a pivot pin 132 and fastened proximally to the plunger holder 138 by plunger's holder fasteners 134,136 (FIG. 6E). Pivot attachment 132 enables low amplitude vertical movement of legs 130 while maintaining the plunger 144 in a stable position during horizontal movement. There is a bolt 144b on proximal end of plunger 144 (FIG. 6D). The bolt 144b fits into the nut 138n within the plunger holder 138 (FIG. 6C). The plunger 144 is connected to the plunger holder 138 by bolt 144b to nut 138n connection.

In one embodiment a small tubing is incorporated into upper distal part of syringe. This vent tube for air removal 141 arises from the opening 145 at the top of syringe 140. Tube 141 has a small 3-way stopcock 143 for air vent tube at its distal end. Tube 141 enables removal of possible air bubbles from the syringe. Distal arcuate fastener 103 is fixed to top plate 102. There are 2 holes 105h on arcuate fastener 103 (FIG. 6C).

FIG. 6D shows syringe 140 with muff 147 and with removable arcuate fastener 107. Muff 147 is fixed integral part of syringe 140 and is made of transparent plastic. There is mechanical stopper 146 for piston on proximal end of syringe. Removable arcuate fastener 107 is placed over the proximal syringe part until it contacts muff 147.

Two rods 105 extent form fastener 107. Syringe 140 as seen in FIG. 6D is placed onto the top plate 102, the tubing length with distal part of syringe is pushed through the arcuate fixed fastener 103 (FIG. 6C). Rods 105 are pushed through the holes 105h and the syringe is fastened by bolt on the rod 105b to screw 105s connection. Proximal part of Plunger 144 is situated into plunger holder 138 and fastened by bolt 144b to nut 138n connection. Detached part of lever is fastened by bolt 122b to nut 123 connection and the syringe pump assembly 100 with lever driving is completed as seen in FIG. 6E.

Arcuate fasteners 103 and 107, rod 105, screw 105s, and the frame parts 102, 104, 106, 108, can be made of light weighed metal or of a firm plastic. Muff 147 should be made of transparent plastic in order to enable visual control of presence of possible air bubbles or presence of possible blood clots within the syringe. The base of frame 106 and vertical arms 104 may have removable connection to top plate 102 which could reduce the portable size of the assembly. Assembly 100 may be provided as a sterile compact unit ready for use, so that no time is needed for set up.

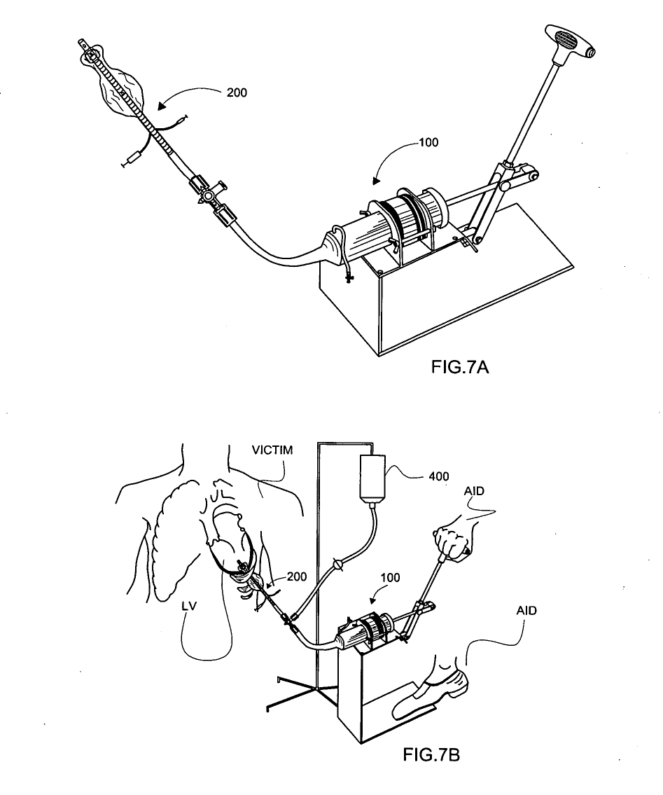

FIG. 7A shows the manual device for cardio circulatory resuscitation in accordance with described embodiment that has syringe pump assembly with facilitated lever mechanism 100 and transthoracic cannula 200.

### Operation

### FIG. 7B

FIG. 7 B shows the device for cardio-circulatory resuscitation in accordance with described alternative embodiment showing the manual actuation facilitated by lever mechanism. A rearward movement of lever 122 effects an aspiration while a forward movement of lever 122 the aspirated blood volume can vigorously be injected back into the chamber of arrested heart. The sidearm of the large bore connector is used for attachment of the fluid infusor 400. In this way an additional volume of fluid can be added in case of reduced aspirating volume; also a hypothermic fluid can be rapidly administered, and any needed medication can be rapidly injected into systemic circulation of a victim.

### Additional Embodiment

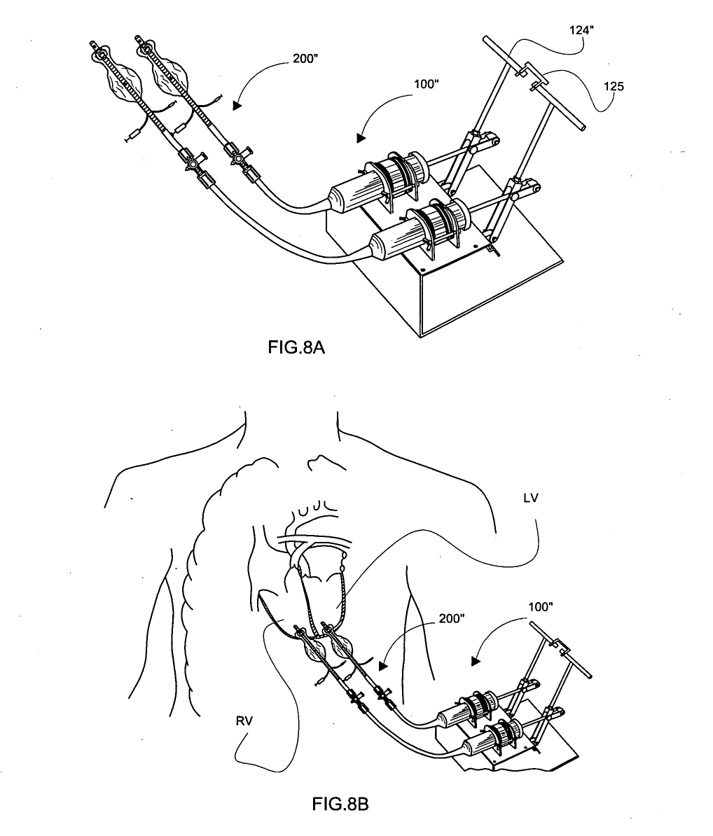

### FIGS. 8A-B

In cases were the resuscitation attempts with utilization of this concept with single access to the left ventricle became prolonged or ineffective, installation of an additional parallel system into the right ventricle could be implemented as shown in FIGS. 8A and 8B.

Two transthoracic access cannulas 200″ are inserted, and connected to double syringe pump assemblies 100″. Both levers 122 are inter connected with the removable connector for common grip 125 to common grip 124″ which enables simultaneous actuation of both parallel installed assemblies providing a replacement of the total heart pumping function. Also, installation of two systems parallel for the right and left heart with individually regulated actuation is possible after removal of grip connector 125.

### Operation

### FIG. 8B

In accordance with described additional embodiment, after installation of the cannula 200 into the left ventricle and connection to pump assembly 100 the same approach is applied for installation of additional parallel situated system 100 and 200 into the right ventricle (FIG. 8B). By manual actuation of common grip 124″ the parallel arranged double pump assembly 100″ provides blood aspiration and injection through the double cannulas 200″. In this way the total (right and left) heart pumping function is replaced. This embodiment can be useful for a prolonged resuscitation need.

### Additional Embodiment

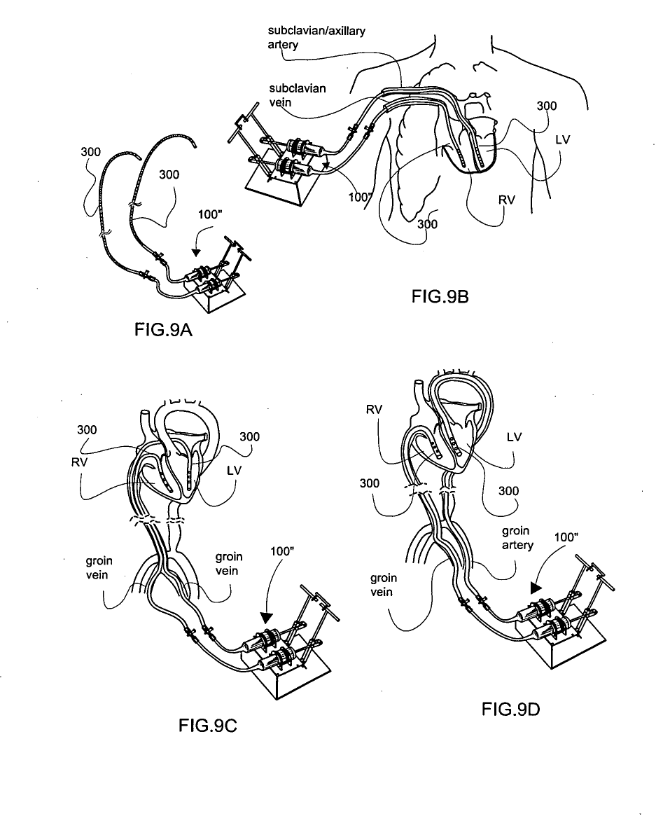

### FIG. 9A

In cases were the resuscitation attempts with utilization of this concept with single access to the left ventricle became prolonged or ineffective, installation of an additional parallel system into the right ventricle could be implemented as shown in FIG. 9A.

Two transvascular access cannulas 300 are connected to double syringe pump assemblies 100″.

### Operation

### FIGS. 9B-D

A subclavian/axillary artery and subclavian vein are punctured (FIG. 9B); transvascular cannulas 300 are placed into the left and right ventricles (LV, RV) and connected to double syringe assembly 100″; by manual actuation the total heart pump function can be replaced.

In accordance with additional embodiment (FIG. 9C) transvacsular cannulas 300 are placed into the right ventricle through the left groin vein and into the left ventricle transseptally through the right groin vein. After connection to double syringe pump assembly 100″ the total heart pump function can be replaced.

This alternative embodiment utilizes an installation through a groin vein by transseptal access through the interatrial septum from the right atrium to the left atrium. This alternative embodiment can be used to treat accidents in a catheterization laboratory environment where there are tools for guiding the procedural activities like ultrasound and fluoroscopy. Also this alternative embodiment can be used in cardiac surgery environment. In some situations after cardiac surgery the patient cannot be disconnected from cardiopulmonary bypass apparatus used during operation. The cannula 300 can be placed from a groin vein transseptally into the left ventricle by direct visual control and the patient can be disconnected from cardiopulmonary bypass. In unstable situations, cannula 300 can be left in place as long as needed. Cannula 300 placed transseptally through a groin vein can be removed without any additional surgical procedure like re-sternotomy or a surgical closure of arterial entry site.

Advantage of such an access is the fact that there is no compromise of arterial circulation. Also in case of cardiac arrest in patients after sternotomy chest compression is problematic.

(FIG. 9D) In accordance with additional embodiment transvacsular cannulas 300 are placed into the right and left ventricle through a groin vein and groin artery respectively. After connection to double syringe pump assembly 100″ the total heart pump function can be replaced.

This alternative embodiment can be used in an in hospital environment where there are tools for guiding the procedural activities like ultrasound and fluoroscopy.

### Alternative Embodiment

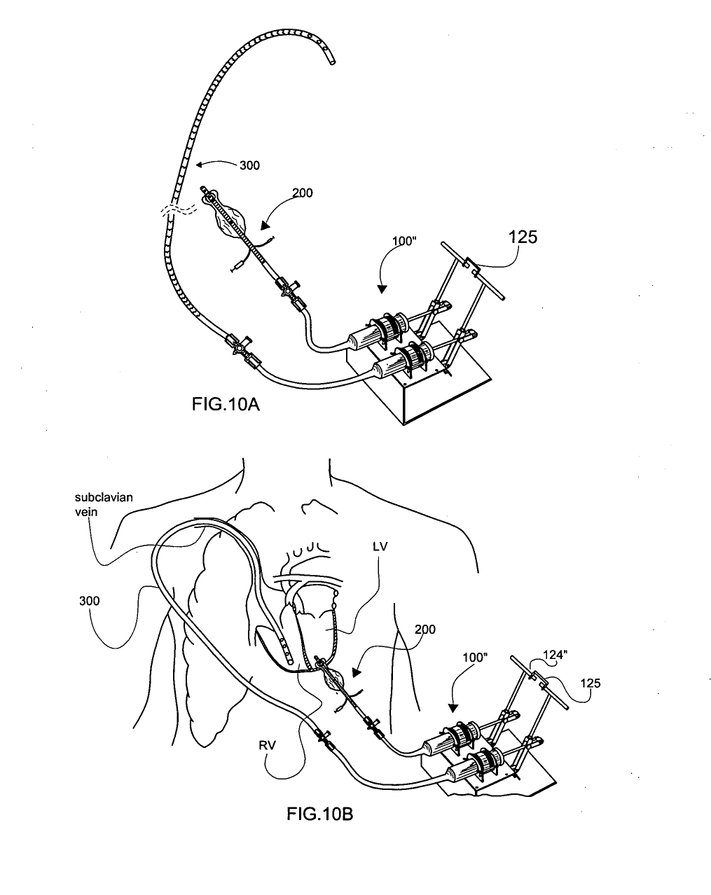

### FIG. 10A

One transvascular cannula 300, and one transthoracic cannula 200 are connected to double syringe pump assembly 100″: having an actuation with manual grips interconnected with removable connector for common grip 125.

### Operation

### FIG. 10B

In accordance with described embodiment, the transthoracic cannula 200 is placed into the left ventricle through the left ventricular apex and a transvascular cannula 300 is placed into the right ventricle through the subclavian vein; the cannulas are connected to double syringe pump assembly 100″; manual actuation of assembly 100″ by common grip 124″ the total heart pump function can be replaced. This embodiment can be used in cases where a prolonged resuscitation is needed.

This embodiment can be used even in an out-of-hospital environment since it can be implemented with a guidance of a portable ultrasound.

### Alternative Embodiment

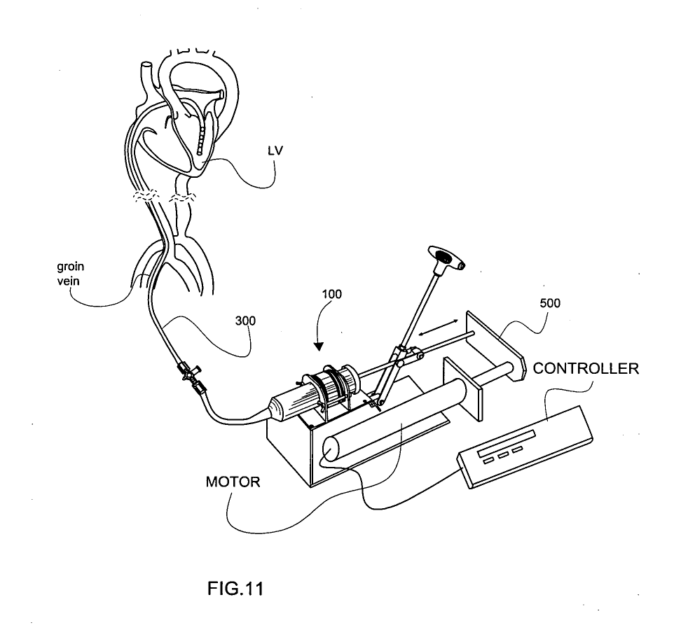

### FIG. 11

The access cannula 300 is placed into the left ventricle and it is attached to syringe pump assembly 100 for providing perfusion in case of cardiac arrest. A motor is attached to the syringe plunger by an removable connector of motor 500 to plunger of the syringe.

### Operation

### FIG. 11

In accordance with this embodiment the manual device for cardio-circulatory resuscitation is combined with a motorized (electro motorized or pneumatic) actuation. In case of prolonged resuscitation a fatigue of an aid can be compensated by utilization of a battery powered motorized mechanism which can be connected to the syringe plunger by a removable connector 500.

### Summarized Description of the Deployment of the Device for Cardio Circulatory Resuscitation

The device described can be used as an alternative to prolonged chest compression or as a last resort measure after failure of today's standard resuscitation attempts. There are large number of victims who are too healthy to be left to die after unsuccessful resuscitation attempt with chest compressions.

If a professional aid has provided an advanced life support to a victim including mechanical ventilation through a tracheal tube, defibrillation attempts, chest compressions, medication injected, and if there is no response, instead of giving up, he takes the described manual device for cardio circulatory resuscitation out of his first aid case and gives the victim an additional chance:

Puncture of the left ventricular apex directly through the chest wall with a vascular needle through the fifth intercostals space at midclavicular line (it takes <1 minute). The puncture does not need any apparatus depended guidance-aspirating a small amount of red colored blood indicates that the left ventricle is punctured; injection of Heparin through the puncturing needle into the left ventricle; placement of a J guide wire 240 through the needle; after removal of puncture needle the transthoracic cannula 200 with introducing dilator 230 (FIG. 2A) is introduced per Seldinger technique into the left ventricle (takes <1 minute); dilator 230 and guide wire 240 are removed, the 3-way stopcock 208 is closed; smaller non compliant balloon 218 is inflated; larger compliant balloon 214 is inflated; cannula 200 is connected to syringe pump assembly 100′ or 100 by stopcock 208 (takes <1 minute) stopcock 208 is redirected and the syringe plunger 144 is moved upwards with both hands or, if the assembly with lever driving is used, the lever 122 is manually moved rearward, which provides aspiration of oxygenated blood from the whole left heart (FIG. 3A); stopcock is re-directed to enable removal of air bubbles if any existing; stopcock 208 is redirected to free the flow from syringe to cannula and the oxygenated aspirated blood is vigorously injected back into the arrested left ventricle (FIG. 3B); aspirations and injections maneuvers are repeated as long as necessary; should there be not enough aspirating blood volume, additional fluid volume can be aspirated from the fluid infusor 400 that can be attached to the side arm of stopcock 208 (FIG. 7B); after restoration of spontaneous circulation the syringe pump 100 is disconnected leaving the cannula in place for supply of additional medication if needed.

After termination of resuscitation the victim can be transported to an institution where the cardiac access site can be closed with a myocardial free wall occluder or surgically.

In an environment with available apparatus-depended guidance like fluoroscopy and or ultrasound the alternative embodiment of this device can be used:

The transvascular cannula assembly (FIG. 4A) is introduced per Seldinger technique through a subclavian/axillary artery (FIG. 5B), or groin artery (FIG. 5C), and cannula 300 is placed into the left ventricle retrograde through aortic valve; cannula 300 is connected to syringe assembly 100′ and the described circulatory support is provided. For a prolonged circulatory support installation of double transthoracic cannulas 200″ (FIG. 8A-B) or double transvascular cannulas 300 (FIG. 9B-D) or 200+300 (FIG. 10B) can be used; both cannulas are connected to double syringe assembly 100″ and by described maneuvers both left and right heart pump functions can be replaced.

### CONCLUSION

Treatment of cardio circulatory arrest has not substantially changed since 50 years. With tracheal intubation, respiratory function is completely replaced with mechanical ventilation while chest compressions are aimed at maintaining perfusion of vital organs. Electrical instability is effectively treated by defibrillations. Today's advanced life support results in return of spontaneous circulation in about 10% of out-of-hospital victims and in 20-30% of in-hospital victims. Only a small number of these primary successfully resuscitated victims survive and even smaller is the number of victims who survive without significant neurological sequels. The dismal low resuscitation success rate is mainly attributable to failure of chest compressions to provide sufficient circulatory flow. Emergency widespread use of surgical cardiopulmonary bypass is not practicable. Percutaneous circulatory assistance like percutaneous left atrial to aorta or left ventricular to aorta bypass, or a miniaturized percutaneous cardiopulmonary bypass (ECMO) are complex, expensive and time consuming.

Non-invasive measures for providing some ventilation during resuscitation attempts had not been sufficiently effective in the past.

Artificial ventilation became effective only after introduction of the procedure with placement of a tubus directly into respiratory system to activate the mechanical function of arrested lung.

Non invasive measures, like chest compressions, for providing some circulation during resuscitation attempts have not been sufficiently effective in the past 50 years.

Artificial circulation will become effective after placement of a tubus directly into heart chamber to activate the mechanical pump function of arrested heart.

The device described in this application could provide sufficient supply of oxygenated blood to vital organs during cardio circulatory arrest. It could be applied in hospital as well as on the field. The device is manually driven independent of any energy power sources, and it could be brought into function rapidly by a professional aid trained for this procedure.

In an emergency situation, such is cardiac arrest, mortality may exceed 90%. Because of large numbers of victims of this condition even small increases in survival could save many lives. Even if the described manual device for cardio circulatory resuscitation would only reduce mortality from 90% to 80%—that would translate into a potential saving of more than 100 000 lives per year in the United States and Europe (some 450 000 people die each year from sudden cardiac arrest in the United States and some 700 000 people die from sudden cardiac arrest each year in Europe—“International Liaison Committee on Resuscitation. Part 2. Adult basic life support. Resuscitation 2005; 67(2/3):187-201”).

From the description above, a number of advantages of some embodiments of my device for cardio circulatory resuscitation become evident:

the device is small, portable, light weighted (<3 kg);
it is simple for manufacturing, relatively less expensive and thus it can be affordable to all economical and social environments;
it can be provided in a ready for use configuration with an instant set up time;
it can be implemented within couple of minutes;
it effects perpetual contractions and distensions of the cavity of an arrested heart chamber by hydraulically transmitted energy which is generated by external manual driving of the syringe pump assembly;
it forces a dead heart to open and close its valves and to contract and distend its wall without spending its own intrinsic energy and without any electrical activity;
it provides effective perfusion with oxygenated blood to vital organs;
it provides heart and pulmonary decompression;
it is independent of any energy power sources;
it can be implemented in-hospital and on the field without any apparatus-depended guidance;
it can be up graded for replacement of the total pumping function of left and right heart by installing double syringe assembly 100″ and double cannulas (FIGS. 8-10);
Application of this device is not limited to resuscitation of victims with cardio-circulatory arrest.

There is possibility to use this device to maintain perfusion of organs in a victim without any chances of survival. This might be the case in a situation where the today's medicine is trying to save victim's organ for donation in those victims who do have a written consent for organ donation in case of inevitable fatal outcome.

Despite the increase in the number of organ transplants there is shortfall of organs suitable for donation. A large number of victims who die from trauma or accidents with organs suitable for transplants could have their organs salvaged. The chances to save the organs of these potential donors in difficult environments are very low due to the fact that there is no system that provides organ perfusion on the field and an organ explantation on the field is not possible. The device presented in this application could be implemented to provide perfusion until a victim with expected fatal outcome is transported to a facility where organ explantation could be performed. The utilization of this device is primarily oriented to human medicine, but the same indications for use are valid for veterinary medicine, i.e. the use of this device is valid for all mammalians.

Although the description above contains many specificities, these should not be construed as limiting the scope of the embodiments but as merely providing illustrations of some of several embodiments.

For example, the manual pumping facilitation can be achieved with a spring mechanism. The syringe plunger 144 is functionally connected to a power spring which would be compressed during manual aspiration phase in which way a considerable potential spring energy could be accumulated and so accumulated energy could reduce energy needed during vigorous injections (not shown).

The frame into which syringe 140 is situated may have different configuration. In one embodiment top plate 102 of the frame may have a removable fastener that could enable a fastening of the syringe to a platform on which a victim rests. In such a configuration (not shown) base of frame 106 and vertical arms 104 would not be needed which would further reduce the size and the weight of the portable device.

The described manually driven pump assembly with a large bore syringe as the pump, is only one of possible embodiments.

In an another embodiment (not shown) the pump may be construed as a compressible bag. Such a bag may be manually compressed for injecting the blood and may be expanded either manually or with an equipped spring system. The volume capacity of such a bag may be appropriately large. Such a bag may be produced from an elastic medical polymer like polyurethane with heparin coated inner surface.

The transthoracic cannula may be equipped with two introducing dilators (not shown); one of which is smaller to be advanced over the guiding wire, and the larger one which is placed over the smaller dilator. The smaller dilator, larger dilator placed over the smaller one, and cannula 200 placed over the larger dilator can be introduced percutaneously as a unit. In this way passage of the assembly through the chest wall might be safer especially in obese victims.

Also, the braided parts of access cannulas (202, or 302) may be manufactured from a memory metal. In this way the braided part of cannula may be expandable after introduction. Such a cannula may have a smaller fixed profile within the passageway through the entry site and an expandable lumen within a blood vessel and within a heart chamber which could increase the flow capacity of such a configuration. Such cannulas are already commercially available, but some configurations could be modified for use as a part of the device described. This version could be useful especially for transvascular cannula 300.

Cannula 300 may have extension of the wires of the braided part of wall.

These wires could make non traumatic arms (not shown) radial expanded distal to end opening 306 of cannula which could provide stable anchorage and prevent dislodgement of the cannula during repeated vigorous injections maneuvers.

The wires could have folding position during introduction and expanding position after placement into heart chamber. Thus the scope of the embodiments should be determined by the appended claims and their legal equivalents, rather than by the examples given.

1. A manual assembly for invasive cardio-circulatory resuscitation comprising
a large volume syringe pump assembly,
a large bore cannula for accessing a chamber of an arrested heart,
means for a rapid connection of said syringe pump assembly to said cannula.

2. The assembly of claim 1 wherein said syringe pump assembly having
a large volume syringe situated into an immobilization frame
wherein said frame having further means for vigorous manual actuation of said syringe pump that enables repeated aspirations and injections of large volume of blood.

3. The assembly of claim 1 wherein said syringe pump assembly comprises
a large volume syringe situated within a cylindrical frame
wherein said frame having means for stabilization of said syringe
wherein said means for stabilization include syringe fasteners of said frame to said syringe wherein said means for stabilization having further frame props for syringe support
wherein said syringe having further on its distal end a tubing length which is incorporated into the distal end of said syringe, wherein said tubing length having on its distal end a connector for a rapid connection to said cannula, wherein said syringe having further a plunger with a large grip for power double handed driving
whereby said device is immobilized during the procedure by holding the foot of the aid on the pedal of said frame.

4. The assembly of claim 1 wherein
said cannula comprising a large bore tubular body that has a part with a wire reinforced wall and a part with a non braided transparent wall wherein said part with non braided wall has a large bore 3-way stopcock incorporated into proximal end of said non braided part.

5. The assembly of claim 4 wherein
said cannula is assembled with means for direct percutaneous trans-thoracic introduction and placement into a chamber of an arrested heart wherein said means for trans-thoracic introduction include a single large introducing dilator for over the wire introduction.

6. The assembly of claim 5 wherein
said cannula having further means for sealing its own passageway from exterior of the body through the chest wall and through the wall of heart chamber of an arrested heart wherein said means for sealing include a large compliant balloon situated on the outer wall of said cannula wherein said balloon is covering the length of said cannula from exterior of the body through the chest wall and chest cavity through the wall of the heart chamber to the side holes on the distal end of said cannula whereby said compliant balloon when inflated makes a waist within its intracorporeal passageway and expands proximal and distal of said passageway.

7. The assembly of claim 6 wherein said cannula having further means for its own stabilization within the passageway and within the chamber of an arrested heart wherein said means for stabilization include a smaller non compliant balloon situated on the outer wall of said cannula within the larger compliant balloon wherein said smaller balloon is covering only the length of said cannula that is situated within the chamber of arrested heart whereby said non compliant balloon together with inflated said large compliant balloon stabilizes said cannula within the heart chamber preventing its dislodgement during vigorous injections of blood or fluid volume through said cannula.

8. The assembly of claim 7 wherein said cannula having further means for a rapid connection to said pump assembly said means for rapid connection comprising a large bore 3-way stopcock incorporated into proximal end of said cannula wherein said 3-way stopcock having a lumen large enough to accept a large introducing dilator.

9. The assembly of claim 1 wherein said syringe pump assembly is equipped with means for facilitated manual driving comprising a lever pivotally attached to the plunger of said syringe and to the frame into which said syringe is situated.

10. The assembly of claim 9
wherein said syringe having further, a small tubing length for air removal incorporated into its upper distal end
wherein said small tubing length for air removal includes further on its distal end a small 3-way stopcock wherein said syringe having further on its outer wall means for engagement of said syringe into an immobilization frame wherein said means for engagement include a muff incorporated into outer wall of said syringe.

11. The assembly of claim 10 wherein
said frame into which said syringe is situated includes a top plate, a base of frame and vertical arms wherein said frame having further fasteners of said syringe to said frame
whereby said fasteners and said muff of said syringe enabling a rapid, installation, fastening, immobilization or removal of said syringe.

12. The assembly of claim 1 comprising:
a) a large volume syringe equipped with means for facilitated manual driving wherein said means for facilitated manual driving include a lever pivotally attached to the plunger of said syringe and to the frame into which said syringe is situated whereby rearward and forward manual movements of said lever moves plunger and piston of said syringe enabling a perpetual aspirations and injections of large volume of blood wherein said syringe having further on its distal end a tubing length wherein said tubing length is incorporated into the distal end of said syringe wherein said tubing length having on its distal end a connector for rapid connection to a large bore 3-way stopcock wherein said syringe having further on its upper distal end an opening for a small tubing length for air removal wherein said small tubing length for air removal includes further on its distal end a small 3-way stopcock comprising further,
b) a large bore cannula for accessing a chamber of an arrested heart wherein said cannula having a distal part that has a wire reinforced wall and a proximal part having a wireless wall wherein said cannula is assembled with means for direct trans-thoracic introduction and placement into the chamber of an arrested heart wherein said means for trans thoracic introduction include an introducing dilator for advancement over a guiding wire wherein said cannula having further means for sealing its own passageway through the chest wall and through the wall of an arrested heart wherein said means for sealing comprising a compliant large balloon situated at the outer wall of said cannula whereby said compliant balloon covers the whole passageway of said cannula from exterior of the body through the chest wall and through the wall of the heart chamber to the interior of the heart chamber of an arrested heart
wherein said cannula having further means for its own stabilization within the chamber of an arrested heart and within its passageway wherein said means for stabilization include a smaller noncompliant balloon situated on the outer wall of the distal part of said cannula within the said larger compliant balloon whereby said noncompliant balloon together with inflated said larger compliant balloon prevents a dislodgement of said cannula during vigorous injections of blood or fluid volume into the arrested heart wherein said cannula having further a large bore 3-way stopcock incorporated into proximal end of said cannula whereby said 3-way stopcock accepts a passage of said introducing dilator during introduction of said cannula whereby said large bore 3-way stopcock enables a rapid connection of said cannula to said syringe assembly
whereby said 3-way stopcock enables further an alternative connection to an infusor for supply of additional fluid volume and for a rapid application of needed medication during and immediately after a resuscitation procedure.

13. The assembly of claim 1 wherein
said cannula having characteristics and means for trans-vascular access to a chamber of an arrested heart wherein said cannula has a longer braided part distally and a non braided shorter part proximally wherein said proximal part having a large bore 3 way stopcock incorporated into proximal end of said proximal part wherein said means for trans-vascular access include a long guiding wire over which a pigtail shaped catheter is advanced, over which catheter an introducing dilator of appropriate dimension is introduced over which dilator said cannula for transvascular access is introduced and placed through a large vein or through a large artery into the chamber of an arrested heart.

14. A manual assembly for cardio-circulatory resuscitation comprising a doubled syringe pump assembly equipped with means for common simultaneous or individual facilitated manual driving, having further double cannulas for accessing both chambers of an arrested heart, having further means for connection of said doubled syringe pump assembly to said double cannulas.

15. The assembly of claim 14 wherein
said doubled cannulas comprise two cannulas for direct trans thoracic placement into chambers of an arrested heart.

16. The assembly of claim 14 wherein
said doubled cannulas comprise two cannulas for trans vascular access to chambers of an arrested heart

17. The device of claim 14 wherein
said doubled cannulas comprise a cannula for trans vascular access to one chamber of an arrested heart and one cannula for trans thoracic access to another chamber of said arrested heart.

18. A method of providing a manually driven rapid perfusion of vital organs during cardio-circulatory arrest comprising:
(a) inserting a large bore cannula into the left ventricle of an arrested heart by percutaneous over the wire technique,
(b) immobilizing said cannula within its passageway
(c) sealing the intracorporeal passageway of said cannula
(d) connecting said cannula to a manually driven large volume syringe pump assembly,
(e) applying a negative suction pressure within said left ventricle by manual actuation of said pump assembly wherein the mitral valve of said left ventricle opens and the aortic valve closes,
(f) aspirating oxygenated blood from the accessed left ventricle and from the left atrium,
(g) injecting a large volume of aspirated blood back into said arrested left ventricle through the same said access cannula whereby the mitral valve closes and the aortic valve opens wherein the injecting volume exceeds the volume capacity of accessed left ventricle whereby the surplus of injecting blood volume is ejected through the aortic valve to perfuse the vital organs,
(h) perpetuating the manually actuated aspiration and injection maneuvers.
